(19) United States
(12) Patent Application Publication
(10) Pub. No.: US 2007/0178176 A1
(43) Pub. Date: Aug. 2, 2007

(54) Composition and method for promoting weight loss

(76) Inventors: Chithan Kandaswami, Lewisville, TX (US); Sidney Stohs, Frisco, TX (US); Christina Coyle, Dallas, TX (US)

Correspondence Address: KRAMER & AMADO, P.C., 1725 DUKE STREET, SUITE 240, ALEXANDRIA, VA, 22314, US

(21) Appl. No.: 11/345,273

(22) Filed: Feb. 2, 2006

(51) Int. Cl. A61K 36/82; A61K 36/537; A61K 31/51; A61K 31/455

(52) U.S. Cl. 424/729; 424/746; 514/276; 514/356

(57) ABSTRACT

A composition for promoting weight loss comprising sage extract, oolong tea extract and guarana extract in concert with thiamine and niacin. The composition is administered to promote weight loss by increasing fat oxidation and/or controlling appetite, thereby promoting weight loss.

### BACKGROUND

Inability to properly manage weight is an increasing problem among the adult population as indicated by increasingly rising rates of obesity. Obesity and excessive weight lead to a litany of health problems such as cardiovascular disease, skeletal and joint disease, diabetes, cancer, poor body image, depression and overall decreased energy. However, maintaining proper body weight is difficult for individuals who lack the time to exercise or the time and knowledge to prepare a healthy diet.

The regulation of body weight involves a complex interaction of the central nervous system (CNS), endocrine system and digestive system. The food consumed from all dietary sources by an individual is the total (caloric) energy intake. The extent of energy (food stores) utilized by the body tissues to perform work (to undertake all the functions of the body) is energy expenditure (EE). The cells utilize energy sources to generate heat, a process known as thermogenesis. All of the energy expended by the body is ultimately converted to heat. The food ingested and the body's metabolic energy stores in the forms of fat (excess calories stored as fat) and glycogen, combine with oxygen and undergo the process of oxidation to generate bioenergy (ATP) in the body cells, utilized for carrying out all life processes. These metabolic processes produce energy as heat (thermogenesis). When one consumes calories in excess of those required for immediate energy production, conversion of a portion of these excess calories to heat occurs, rather than its storage as fat, a process known as diet-induced thermogenesis. The two fundamental approaches to induce weight loss are either to decrease energy (food) intake or to increase energy expenditure by enhanced oxidation of food sources, or a combination of both.

The body possesses an expanded capacity to store fat. The fat stores in the body readily accommodate increasing levels of energy intake over and above those required for daily energy needs. The mobilization of stored fat from the body, in the form of triglycerides, and their hydrolysis to free fatty acids (lipolysis) provides substrates for metabolic oxidation. Raising metabolism, particularly that of fat, utilizes more food reserves and, therefore, results in higher EE. The preferential deployment of fat for enhanced oxidation accelerates the utilization of body fat, thereby depleting fat stores in the tissues and inducing weight loss. In case there is a deficit in the supply of energy, the body draws from fats stored from excess energy intake (from foods) and oxidizes the same (loss of weight).

The body's physiological systems maintain homeostasis with respect to energy (caloric) intake and energy utilization. The regulation of energy balance involves a complex network of hormonal and neural mechanisms. A net imbalance between energy intake and energy expenditure over time impairs body weight maintenance, resulting in weight gain leading to excessive body weight and eventually contributing to obesity, depending on the severity.

When the body encounters an energy deficit in the course of weight-reducing diets, it tends to save energy by reducing thermogenesis. This factor contributes to a failure in sustaining body weight after body weight loss. These circumstances require continually stimulated thermogenesis. After losing weight for a short duration, an individual's weight stabilizes. In order to maintain body weight there is a need to control food intake and stimulate thermogenesis on an ongoing basis.

Body weight control methods often yield short-term positive results and sustained weight maintenance is a real challenge. The difficult task is to identify substances, which are readily able to suppress appetite and reduce food (energy) intake, and continuously maintain increased metabolism and enhanced EE, thereby facilitating body weight maintenance.

Prior art attempts at increasing energy expenditure have primarily used large amounts of stimulants such as caffeine and ephedra. Caffeine in particular has been a favored CNS stimulant used in weight management. While caffeine at high doses increases energy expenditure and induces fat oxidation, continued or sustained weight loss becomes a major concern where greater amounts of caffeine lead to a gradual habituation and eventual decrease in efficacy. Furthermore, stimulants such as caffeine induce possible transient side effects on the cardiovascular system. Cardiovascular side effects are often a concern for those with unhealthy body weight where obesity and cardiovascular health problems often overlap. Caffeine should also be avoided by people who suffer from kidney disease, an overactive thyroid, a tendency to spasms, or problems with anxiety or panic.

Accordingly, there is a need for providing a composition for promoting weight loss, through increased fat oxidation and modulation of appetite that contains decreased levels of caffeine. The composition should provide weight loss effects without the drawbacks of caffeine habituation leading to decreased efficacy over time and cardiovascular and CNS side effects caused by high amounts of caffeine. Furthermore, there is a need for methods for promoting weight loss by administering a composition that increases fat oxidation and modulates appetite with decreased levels of caffeine.

### SUMMARY

According to an embodiment, a composition comprises sage extract, oolong tea extract, guarana extract, thiamine and niacin. In another embodiment a method comprises promoting weight loss by increasing fat oxidation and/or decreasing appetite by administering a pharmaceutical composition comprising standardized sage extract, standardized oolong tea extract, standardized guarana extract, thiamine and niacin. The combination of the elements of the composition promotes weight loss while administering decreased amounts of caffeine as compared to conventional formulations.

### DETAILED DESCRIPTION

For simplicity and illustrative purposes, the principles are shown by way of examples of compositions and methods described. In the following description, numerous specific details are set forth in order to provide a thorough understanding of the examples. It will be apparent however, to one of ordinary skill in the art, that the examples may be practiced without limitation to these specific details. In other instances, well known methods and compositions are not described in detail so as not to unnecessarily obscure understanding of the examples.

In an example of a composition for promoting weight loss, the composition promotes fat oxidation and controls appetite while applying limited levels of caffeine. In an embodiment, a composition comprises a combination of oolong tea extract, sage extract, guarana extract, thiamine and niacin. The combination of all of the elements provides for weight loss with decreased amounts of caffeine.

Oolong tea extracts are derived from natural tea produced from the leaves of Camellia sinensis of the genus Theaceae. Tea products are manufactured first by rolling and steaming of tender tea leaves to prepare green tea. The green tea is then fermented through the oxidative transformation of tea leaf phenolics, involving natural browning reactions induced by oxidizing enzymes (polyphenol oxidases) within the plant cell. The green tea is fully fermented into black tea. Oolong tea is partially fermented tea that contains both native polyphenols and oxidized polyphenols as a result of the partially completed fermentation process. Therefore, oolong teas have a unique blend of natural and oxidized tea polyphenols.

The components of oolong tea display intestinal lipase inhibitory and thermogenic properties. Oolong tea constituents also appear to promote hormone sensitive lipase activity in adipose tissues resulting in lipolysis and have the ability to influence the sympathoadrenal system and the turn over of catecholamines such as epinephrine and norepinephrine. The modulation of catecholamine turn over influences circulating and neuronal catecholamines, which increase metabolic rate and fat oxidation.

The oolong tea extract may be standardized to contain particular concentrations of chemical components such as native polyphenols, oxidized polyphenols and caffeine. While the polyphenols and caffeine of the oolong tea extract are part of the total amount of polyphenols and total amount of caffeine of the composition, the standardized extract provides qualitatively and quantitatively different constituents than those provided by additional elements of the composition, such as sage extract and guarana extract. According to an embodiment, oolong tea extract may be standardized to comprise not less than about 40% polyphenols. In another embodiment, the oolong tea extract polyphenols are part of the total amount of polyphenols in the composition and the oolong tea extract caffeine is part of the total amount of caffeine in the composition.

Sage extract is derived from the leaf of Salvia officinalis. The active ingredients in sage have a number of effects on the CNS. The polyphenols and related aromatic ingredients in the sage extract modulate the gamma-aminobutyric acid (GABA) system and the benzodiazepine receptor binding sites, which influence satiety, appetite, food intake, energy expenditure and energy balance. GABA plays an important role in controlling energy balance within the central nervous system and food intake. Sage extracts also influence the function of NMDA (N-methyl-D-aspartic acid) receptors. NMDA receptors contribute to systems controlling natural feeding and body weight regulation.

Additionally, sage extracts reduce body weight gain and accumulation of epididymal fat weight by suppressing pancreatic lipase activity and fat accretion. Other components reduce nuclear factor-kappaB, a transcription factor involved in obesity.

The sage extract may be standardized to contain particular concentrations of chemical components such as sage polyphenols. While the polyphenols of the sage tea extract are part of the total amount of polyphenols, the standardized extract provides qualitatively and quantitatively different constituents than those provided by additional elements of the composition, such as the oolong tea extract. According to an embodiment, sage extract may be standardized to contain not less than about 10% polyphenols. In another embodiment, the sage extract polyphenols are part of the total amount of polyphenols in the composition.

Guarana extract is derived from the seed of Paullina cupana, a tropical plant found in the Amazon basin. The seeds of Paullina cupana are peeled, dried, roasted, ground much like coffee beans. Guarana extracts provide concentrated amounts of caffeine in addition to theobromine and theophylline. Caffeine related compounds derived from guarana extract are often also known as guararine. The amounts of guarana extract in the composition are limited to decrease total caffeine.

The guarana extract may be standardized to contain particular concentrations of chemical components such as polyphenols and caffeine. While the polyphenols and caffeine of the guarana extract are part of the total amount of polyphenols and total amount of caffeine of the composition, the standardized extract provides qualitatively and quantitatively different constituents than those provided by additional elements of the composition, such as sage extract and oolong tea extract. According to an embodiment, guarana extract may be standardized to comprise not less than about 50% caffeine. In another embodiment, the guarana extract caffeine is part of the total amount of caffeine in the composition and the guarana extract polyphenols are part of the total amount of polyphenols in the composition.

According an embodiment, a composition comprises oolong tea extract standardized to comprise not less than 40% polyphenols, sage extract standardized to comprise not less than 10% polyphenols and guarana extract standardized to comprise not less than about 50% caffeine. According to another embodiment, the ratio of total amount of polyphenols to total amount of caffeine in the composition is in the range of about 4:1 to about 1.2:1.

In another embodiment, the composition may comprise a weight ratio of sage extract to oolong tea extract in the range from about 5:1 to about 3:4. The composition may comprise a weight ratio of sage extract to guarana extract in the range from about 6:1 to about 1.5:1. The composition may alternatively comprise a weight ratio of oolong tea extract to guarana extract in the range from about 5:1 to about 1:2. According to another embodiment, the weight ratio of the sum of sage extract and oolong tea extract to guarana extract is in the range from about 10:1 to about 2:1.

The term “standardized” as used in the field of naturally derived nutritional products refers to the process for delivering a product with a specific minimum concentration of one or more plant constituents. Standardization represents the level of concentration of particularly desired elements from a plant source. Methods for standardizing plant substances, including oolong tea, sage and guarana extracts, are well known in the art. Furthermore, the measurement of particular plant constituents on which standardization is based is also well known in the art. In an example, oolong tea extract standardization may be performed by UV spectrophotometry using appropriate standards and sage extract may be measured by any number of spectrophotometric assays. Other methods of measurement include chromatography, such as thin layer chromatography, high pressure liquid chromatography, gas chromatography and liquid chromatography-mass spectrometry.

The term “about” when used as a modifier of a numerical range or amount designates an approximation of the range or the amount whereby minor deviations from the range or amount are within the scope of the invention. Such deviations are known in the art of manufacturing formulations having a number of different ingredients of varying weight and consistency.

According to an embodiment, a composition contains standardized sage extract in the amount of about 30-1000 mg; standardized oolong tea extract in the amount of about 40-1000 mg; and standardized guarana extract in the amount of about 20-600 mg.

A composition may be administered on the basis of a serving size. A serving size designates a fixed amount of the composition, whereby multiple servings or fractions of servings may be administered to provide weight loss through fat oxidation and/or appetite control.

According to an embodiment, a composition comprises; standardized sage extract in the amount of about 300-500 mg per serving; standardized oolong tea extract in the amount of about 100-400 mg per serving; and standardized guarana extract in the amount of about 90-150 mg per serving.

The compositions may contain limited amounts of caffeine per serving size. In an example, the composition may include less than 100 mg of caffeine per serving size. In an alternative embodiment, the amount of caffeine per serving size may be less than 60 mg per serving.

Thiamine, also known as vitamin B₁, is a water-soluble vitamin having a structure that consists of a substituted pyrimidine ring joined by a methylene bridge to a substituted thiazole ring. Studies have shown that the active form of thiamine plays an essential role as a coenzyme/cofactor in key reactions in carbohydrate metabolism and thermogenesis generally. It is also involved in the metabolism of branched-chain amino acids, and may have non-coenzyme (non-cofactor) roles in excitable cells. According to an embodiment, thiamine may be in the form of thiamine hydrochloride (“thiamine HCl”). In an example, thiamine HCl may be assayed at purity of 98.5% or above.

Niacin, also known as nicotinic acid or vitamin B₃, has the chemical name pyridine-3-carboxylic acid. The vitamin, via its active forms, is involved in a wide range of biological processes, including the production of energy, the synthesis of fatty acids, cholesterol and steroids, signal transduction, the regulation of gene expression and the maintenance of genomic integrity. Pharmaceutical doses are used as an anti-hyperlipidemic agent. According to an embodiment, niacin may be in the form of niacinamide. In an example, niacin may be assayed at purity of 98% or above.

According to an embodiment, a composition contains thiamine in the amount of about 0.2-12 mg; niacin in the amount of about 1-60 mg; sage extract in the amount of about 30-1000 mg; oolong tea extract in the amount of about 40-1000 mg; and guarana extract in the amount of about 20-600 mg.

According to an embodiment, a composition comprises thiamine in the amount of about 3-5 mg per serving; niacin in the amount of about 20-40 mg per serving; sage extract in the amount of about 300-500 mg per serving; oolong tea extract in the amount of about 100-400 mg per serving; and guarana extract in the amount of about 90-150 mg per serving. The compositions may contain limited amounts of caffeine per serving size. In an example, the composition may include less than 100 mg of caffeine per serving size. In an alternative embodiment, the amount of caffeine per serving size may be less than 60 mg per serving.

In an embodiment, the composition may contain further ingredients including, but not limited, to L-glutamine, chlorogenic acid, green coffee extract, lysine, inositol, choline, vitamin A, calcium, magnesium, potassium vitamin C, quercetin-3-rhamnoside (quercitrin) or quercetin-3-glucoside (isoquercitrin) and combinations thereof.

According to an embodiment, a serving size of the composition contains thiamine in the amount of 3-5 mg; niacin in the amount of about 20-40 mg; sage extract in the amount of about 300-500 mg; oolong tea extract in the amount of about 100-400 mg; guarana extract in the amount of about 90-150 mg; and one or more of the following ingredients: L-glutamine in the amount of about 300-500 mg; chlorogenic acid in the amount of about 100-300 mg; green coffee extract in the amount of about 100-300 mg; lysine in the amount of about 200-400 mg; inositol in the amount of about 20-50 mg; choline in the amount of about 20-40 mg; vitamin A in the amount of about 1,000-3,000 IU; magnesium in the amount of about 50-150 mg; calcium in the amount of about 100-300 mg; potassium in the amount of about 50-150 mg; vitamin C in the amount of about 40-120 mg; and quercitrin in the amount of about 60-180 mg.

A composition may further include a number of non-active compounds, such as diluents, buffers, preservatives, desiccants, thickeners, fillers, flavorings, sweeteners, colorings, effervescents and any other excipients or non-active ingredients known in the art. The composition may be administered as a number of dosage forms, including, but not limited to powders, liquids, suspensions, capsules, tablets, caplets or chewing gums and/or may be formed as part of a food product. In an example, the composition is in the form of a tablet, capsule or caplet for oral administration.

An alternative embodiment comprises a method for increasing fat oxidation by administering a composition comprising standardized sage extract, standardized oolong tea extract and standardized guarana extract. According to the method, the composition may be administered in an oral dosage form twice a day, each oral dosage form containing one serving size of the composition. According to an alternative embodiment, the composition may be administered in one serving about thirty minutes before breakfast and in a second serving about thirty minutes before lunch. The composition may also be administered in combination with a number of other nutritional supplement products.

According to an embodiment, the method for administering the composition increases fat oxidation through affecting the sympathoadrenal system. In particular, the combination of polyphenols, from oolong tea extract, sage extract and guarana extract, and caffeine, from guarana extract and oolong tea extract, has the ability to influence the sympathoadrenal system and the turn over of catecholamines such as epinephrine and norepinephrine.

Polyphenols, related to the compounds found in tea extracts and sage extracts, have been shown to elevate endogenous levels of catecholamines. The modulation of catecholamine turn over influences circulating and neuronal catecholamines, which increase metabolic rate and fat oxidation. The maintenance of levels of the intracellular signaling molecule, cyclic AMP (cAMP), in the sympathetically innervated cells (such as cells of the adipose tissue, repository of fat storage) is critical for increasing metabolism and enhancing energy expenditure by the metabolism of fats. Catecholamine stimulation of the sympathetic nervous system has been shown to increase plasma norepinephrine levels by 27% and fat oxidation by 72%. An alternative embodiment comprises a method for controlling appetite. According to the method, a composition comprising standardized sage extract, standardized oolong tea extract and standardized guarana extract may be administered in an oral dosage form to control appetite. Additional elements may include thiamine and niacin. The oral dosage form preferably contains a single serving and may be administered twice a day. In an alternative embodiment, an oral dosage form containing one serving of the composition may be administered once about thirty minutes before breakfast and then again about thirty minutes before lunch.

According to an embodiment, administration of the combination of constituents results in controlling appetite. Appetite may be controlled by either being decreased or maintained at a constant level without increase. When taken together, sage polyphenols and oolong tea polyphenols, in concert with other oolong tea constituents, synergistically diminish food consumption. Additionally, the combination of tea polyphenols, non-polyphenol oolong tea constituents and sage polyphenols results in the modulation of gamma-aminobutyric acid (“GABA”) receptors and benzodiazepine binding sites which regulate appetite and food intake. GABA (gamma-aminobutyric acid, the major inhibitory neurotransmitter in the brain), receptors and benzodiazepine binding sites in these receptors modulate appetite and food intake. Sage polyphenols in particular show affinity to human benzodiazepine receptors. The complex interactions between several neurotransmitters such as GABA, dopamine, serotonin, neuropeptide Y, leptin, acetylcholine, melanin-concentrating hormone, nitric oxide, cytokines, insulin and insulin receptors in the brain ultimately determine and regulate food intake. The composition affects appetite through modulation of GABA-benzodiazepine receptor sites.

Sage extracts also influence the function of NMDA (N-methyl-D-aspartic acid) receptors. NMDA receptors contribute to some forms of natural feeding and body weight regulation. The mixture of polyphenols in the composition may impair food intake by this action.

Example: The following table is an example of an embodiment of a composition in a serving size:

### EXAMPLE 1

| INGREDIENTS | AMOUNT PER SERVING | |
|---|---|---|
| Thiamine (as HCl) | 3 | mg |
| Niacin (as niacinamide) | 20 | mg |
| Sage extract (leaf-Salvia officinalis) | 300 | mg |
| Oolong tea extract (leaf-Camellia sinensis) | 200 | mg |
| Guarana extract (seed-Paullinia cupana) | 90 | mg |

The formulation of the Example was used in a comparison study to determine its effectiveness versus a conventional weight-loss formulation characterized by high levels of caffeine versus polyphenols. The conventional formulation is described below:

Conventional Weight-Loss Formulation

| | AMOUNT PER | |
|---|---|---|
| INGREDIENTS | SERVNG | |
| Vitamin A (as beta-carotene) | 1,000 | IU |
| Niacin | 20 | mg |
| Magnesium (as magnesium phosphate) | 25 | mg |
| Potassium (as potassium phosphate) | 50 | mg |
| Bitter orange extract (pericarp-Citrus aurantium) | 500 | mg |
| Guarana extract (seed-Paullinia cupana) | 200 | mg |
| Green tea extract (leaf-Camellia sinensis) | 10 | mg |

The conventional weight-loss formulation is generally administered as part of a more extensive nutritional supplement and multivitamin regimen. Therefore, Example 1 was also administered with the multivitamin and nutritional supplement regimen to obtain an accurate comparison with the conventional formulation. While Example 1 above was used in combination with additional nutritional supplements, an alternative embodiment may be administering alone or in combination with other nutritional supplements.

The additional nutritional supplements administered in combination with Example 1 and the conventional weight-loss formulation, for the purposes of comparison testing, are described below. The supplements include a Multiple Vitamin and Mineral Supplement, an Omega-3 Fatty Acid Supplement, a Multinutrient Supplement, a Probiotic Dietary Supplement, and a Citrus Dietary Supplement.

Multiple Vitamin and Mineral Supplement

| INGREDIENTS | AMOUNT PER SERVING | |
|---|---|---|
| Vitamin A (as palmitate) | 2,500 | IU |
| Vitamin A (as beta-carotene) | 12,500 | IU |
| Vitamin C (as ascorbic acid) | 600 | mg |
| Vitamin D (as cholecalciferol) | 400 | IU |
| Vitamin E (as d-alpha tocopheryl succinate) | 150 | IU |
| Thiamine (as thiamine HCl) | 4.5 | mg |
| Riboflavin | 5.1 | mg |
| Niacin (as niacinamide/niacin) | 60 | mg |
| Vitamin B-6 (pyridoxine HCl) | 6 | mg |
| Folic Acid | 800 | mcg |
| Vitamin B-12 (as cyanocobalamin) | 36 | mcg |
| Biotin | 300 | mcg |
| Pantothenic acid | 30 | mg |
| Calcium (as amino acid chelate) | 150 | mg |
| Phosphorus (as amino acid chelate) | 25 | mg |
| Iodine (from kelp) | 150 | mcg |
| Magnesium (as amino acid chelate) | 175 | mg |
| Zinc (as zinc monomethionine) | 15 | mg |
| Selenium (L-selenomethionine) | 80 | mcg |
| Copper (as amino acid chelate) | 2 | mg |
| Manganese (as amino acid chelate) | 4 | mg |
| Chromium (as chromium citrate) | 100 | mcg |
| Molybdenum (as amino acid chelate) | 50 | mcg |
| Potassium (as potassium chloride and amino | 100 | mg |
| acid chelate) | | |
| Inositol | 6 | mg |
| Choline (as bitartrate) | 60 | mg |
| Boron (as amino acid chelate) | 300 | mcg |
| Vanadium (as bis-maltolato-oxovanadium) | 50 | mcg |
| Silicon (as amino acid chelate) | 500 | mcg |
| Coenzyme Q-10 | 150 | mcg |
| Octacosanol | 2 | mg |
| Ribonucleic acid (RNA) | 2 | mg |
| Garlic powder, odorless (bulb-Allium | 50 | mg |
| sativum) | | |
| L-Glutathione | 5 | mg |
| Citrus flavonoids | 100 | mg |
| Grape extract (seed-Vitis vinifera) | 5 | mg |
| Milk thistle extract (seed-Silybum marianum) | 5 | mg |
| Ginkgo extract (leaf-Ginkgo biloba) | 10 | mg |

Omega-3 Fatty Acid Supplement

| INGREDIENTS | AMOUNT PER SERVING | |
|---|---|---|
| Vitamin E (as d-alpha tocopheryl acetate) | 3 | IU |
| Eicosapentaenoic Acid (from marine lipids) | 300 | mg |
| Docosahexaenoic Acid (from marine lipids) | 200 | mg |

Multinutrient Supplement

| INGREDIENTS | AMOUNT PER SERVING | |
|---|---|---|
| Vitamin C (as magnesium/sodium/potassium | 300 | mg |
| ascorbates) | | |
| Vitamin B-6 (as pyridoxine HCl) | 2 | mg |
| Iodine (as potassium iodide) | 25 | mcg |
| Magnesium (as magnesium ascorbate) | 10 | mg |
| Zinc (as zinc monomethionine) | 1 | mg |
| Chromium (as citrate) | 50 | mcg |
| Garcinia extract (fruit-Garcinia cambogia) | 1,000 | mg |
| Oolong tea extract (leaf-Camellia sinensis) | 100 | mg |
| Guarana extract (seed-Paullinia cupana) | 75 | mg |
| Eleuthero extract (root-Eleutherococcus | 25 | mg |
| senticosus) | | |
| Tulsi extract (leaf-Ocimum sanctum) | 25 | mg |
| Taurine | 25 | mg |
| Beta-sitosterol | 12.5 | mg |
| L-Carnitine (as tartrate) | 12.5 | mg |
| Gymnema extract (leaf-Gymnema sylvestre) | 5 | mg |
| Vanadium (as bis-maltolato-oxovanadium) | 100 | mcg |

Probiotic Supplement

| INGREDIENTS | AMOUNT PER SERVING | |
|---|---|---|
| Vitamin A (as beta-carotene) | 1,000 | IU |
| Zinc (as zinc monomethionine) | 500 | mcg |
| Lactobacillus acidophilus | 0.5 | Billion |
| Bifidobacterium bifidum | 0.5 | Billion |
| Fructooligosaccharides | 250 | mg |
| Moomiyo | 25 | mg |
| Artichoke extract (leaf-Cynara scolymus) | 250 | mg |

Citrus Dietary Supplement

| INGREDENTS | AMOUNT PER SERVING | |
|---|---|---|
| D-Limonene | 60 | mg |
| Grapefruit juice powder | 200 | mg |
| 5-Hydroxytryptophan (seed-Griffonia | 25 | mg |
| simplicifolia) | | |

The comparison testing was performed in two cohorts. In the first study, a total of 42 participants who were previously taking the conventional weight-loss formulation were administered Example 1. Example 1 replaced the conventional formulation in the nutritional supplement regimen. A serving of Example 1 was administered 30 minutes before breakfast and again 30 minutes before lunch. The remaining supplement formulations were administered with a meal, preferably breakfast, except for the probiotic supplement and the multinutrient supplement which were administered at the same time as Example 1.

Of the total number of participants in the first study, the mean weight loss was 1.6 pounds over the course of 14 days. The results of the study are shown as Graph 1 below. According to the first bar of Graph 1, 62% of the participants rated Example 1 to be most effective for appetite control as compared to the conventional weight-loss formulation. 45% of the participants rated Example 1 to be most effective for increasing energy as compared to the conventional weight-loss formulation with 31% of the participants rating the two products to be equally effective. Overall, 54% of the participants rated Example 1 to be superior to the conventional weight-loss formulation and 23% rated the two to be equivalent.

[image]

A second study was performed on 36 participants following the same protocol. The conventional weight-loss formulation was replaced with Example 1 which was administered according the same nutritional supplement regimen.

The results of the second study are described in Graph 2. Of the total number of participants, the mean weight loss was 0.9 pounds. Of the participants who lost weight, the mean weight loss was 2.5 pounds. As seen in Graph 2, 74% of the participants rated Example 1 to be more effective for appetite control. While 49% of the participants rated Example 1 to be more effective for increasing energy, 40% rated Example 1 to be equally effective as compared to the conventional weight-loss formulation. 64% of the participants rated Example 1 to be more effective in providing a longer duration of increased energy over the conventional weight-loss formulation. Overall, 61% of the participants preferred Example 1 over the conventional formulation and 33% of the participants found the products to be equally effective.

[image]

The findings of the study show that the participants found Example 1 to be more or equally effective in controlling appetite and increasing energy as compared to the conventional weight-loss formulation. The results are surprising when considering the fact that the example formulation contained a significantly reduced amount of caffeine, contrary to conventional thinking in the art. Thereby an embodiment showed superior or at least equal efficacy with decreased amounts of stimulant and therefore decreased risk of habituation and side effects. According to an embodiment, the composition may be administered as a stand-alone product either before meals or at any other time during the day.

Although the present invention has been described in detail with particular reference to preferred embodiments thereof, it should be understood that the invention is capable of other different embodiments, and its details are capable of modifications in various obvious respects. As is readily apparent to those skilled in the art, variations and modifications may be affected while remaining within the spirit and scope of the invention. Accordingly, the foregoing disclosure, description, and figures are for illustrative purposes only, and do not in any way limit the invention, which is defined only by the claims.

What is claimed is:

1. A composition for promoting weight loss comprising:
Sage extract
Oolong tea extract;
Guarana extract;
Thiamine; and
Niacin.

2. The composition of claim 1 wherein the sage extract is standardized to comprise not less than about 10% sage polyphenols.

3. The composition of claim 1 wherein the oolong tea extract is standardized to comprise not less than about 40% oolong tea polyphenols.

4. The composition of claim 1 wherein the guarana extract is standardized to comprise not less than about 50% caffeine.

5. The composition of claim 1 wherein the composition further comprises:
sage extract standardized to comprise not less than about 10% sage polyphenols;
oolong tea extract standardized to comprise not less than about 40% oolong tea polyphenols; and
guarana extract standardized to comprise not less than about 50% caffeine.

6. The composition of claim 5 wherein a ratio of total amount of polyphenols in the composition to total amount of caffeine in the composition is in a range from about 4:1 to about 1.2:1.

7. The composition of claim 5 wherein a ratio of weight of sage extract to weight of oolong tea extract is in a range from about 5:1 to about 3:4.

8. The composition of claim 5 wherein a ratio of weight of sage extract to weight of guarana extract is in a range from about 6:1 to about 1.5:1.

9. The composition of claim 5 wherein a ratio of weight of oolong tea extract to weight of guarana extract is in a range from about 5:1 to about 1:2.

10. The composition of claim 5 wherein a weight ratio of the sum of the weight of sage extract and the weight of oolong tea extract to the weight of guarana extract is in a range from about 10:1 to about 2:1.

11. The composition of claim 5 wherein the composition further comprises:
Sage extract in the amount of 300-500 mg;
Oolong tea extract in the amount of 100-400 mg;
Guarana extract in the amount of 90-200 mg;
Thiamine in the amount of 3-5 mg;
Niacin in the amount of 20-40 mg.

12. The composition of claim 11 wherein the composition comprises about 1-200 mg of caffeine.

13. The composition of claim 12 wherein the composition comprises about 1-60 mg of caffeine.

14. The composition of claim 1 further comprising at least one of L-glutamine, chlorogenic acid, green coffee extract, lysine, inositol, choline, vitamin A, calcium, magnesium, potassium, vitamin C and quercitrin.

15. A method for promoting weight loss comprising:
orally administering an effective amount of a composition to a mammal wherein the composition comprises:
Sage extract;
Oolong tea extract;
Guarana extract;
Thiamine; and
Niacin;
wherein the composition controls the appetite of the mammal.

16. The method of claim 15 wherein the sage extract is standardized to comprise not less than about 10% sage polyphenols.

17. The method of claim 15, wherein the oolong tea extract is standardized to comprise not less than about 40% oolong tea polyphenols.

18. The method of claim 15 wherein the guarana extract is standardized to comprise not less than about 50% caffeine.

19. The method of claim 15 wherein the composition further comprises:
sage extract standardized to comprise not less than about 10% sage polyphenols;
oolong tea extract standardized to comprise not less than about 40% oolong tea polyphenols; and
guarana extract standardized to comprise not less than about 50% caffeine.

20. The method of claim 19 wherein a ratio of total amount of polyphenols in the composition to total amount of caffeine in the composition is in a range from about 4:1 to about 1.2:1.

21. The method of claim 19 wherein a ratio of weight of sage extract to weight of oolong tea extract is in a range from about 5:1 to about 3:4.

22. The method of claim 19 wherein a ratio of weight of sage extract to weight of guarana extract is in a range from about 6:1 to about 1.5:1.

23. The method of claim 19 wherein a ratio of weight of oolong tea extract to weight of guarana extract is in a range from about 5:1 to about 1:2.

24. The method of claim 19 wherein a weight ratio of the sum of the weight of sage extract and the weight of oolong tea extract to the weight of guarana extract is in a range from about 10:1 to about 2:1.

25. The method of claim 19 wherein the composition further comprises:
Sage extract in the amount of 300-500 mg;
Oolong tea extract in the amount of 100-400 mg;
Guarana extract in the amount of 90-200 mg;
Thiamine in the amount of 3-5 mg;
Niacin in the amount of 20-40 mg.

26. The method of claim 25 wherein the composition comprises about 1-200 mg of caffeine.

27. The method of claim 26 wherein the composition comprises about 1-60 mg of caffeine.

28. The method of claim 19 further comprising administering the composition to the mammal at least about 30 minutes before the mammal eats a meal.

29. The method of claim 19 further comprising controlling appetite by decreasing the appetite of the mammal.

30. The method of claim 19 further comprising controlling appetite by maintaining the appetite of the mammal at a constant level.

31. The method of claim 15 wherein the composition further comprises at least one of L-glutamine, chlorogenic acid, green coffee extract, lysine, inositol, choline, vitamin A, calcium, magnesium, potassium, vitamin C and quercitrin.

32. A method for promoting weight loss comprising:
orally administering an effective amount of a composition to a mammal wherein the composition comprises:
Sage extract;
Oolong tea extract;
Guarana extract;
Thiamine; and
Niacin;
wherein the composition increases fat oxidation by the mammal.

33. The method of claim 32 wherein the sage extract is standardized to comprise not less than about 10% sage polyphenols.

34. The method of claim 32, wherein the oolong tea extract is standardized to comprise not less than about 40% oolong tea polyphenols.

35. The method of claim 32 wherein the guarana extract is standardized to comprise not less than about 50% caffeine.

36. The method of claim 32 wherein the composition further comprises:
sage extract standardized to comprise not less than about 10% sage polyphenols;
oolong tea extract standardized to comprise not less than about 40% oolong tea polyphenols; and
guarana extract standardized to comprise not less than about 50% caffeine.

37. The method of claim 36 wherein a ratio of total amount of polyphenols in the composition to total amount of caffeine in the composition is in a range from about 4:1 to about 1.2:1.

38. The method of claim 36 wherein a ratio of weight of sage extract to weight of oolong tea extract is in a range from about 5:1 to about 3:4.

39. The method of claim 36 wherein a ratio of weight of sage extract to weight of guarana extract is in a range from about 6:1 to about 1.5:1.

40. The method of claim 36 wherein a ratio of weight of oolong tea extract to weight of guarana extract is in a range from about 5:1 to about 1:2.

41. The method of claim 36 wherein a weight ratio of the sum of the weight of sage extract and the weight of oolong tea extract to the weight of guarana extract is in a range from about 10:1 to about 2:1.

42. The method of claim 36 wherein the composition further comprises:
Sage extract in the amount of 300-500 mg;
Oolong tea extract in the amount of 100-400 mg;
Guarana extract in the amount of 90-200 mg;
Thiamine in the amount of 3-5 mg;
Niacin in the amount of 20-40 mg.

43. The method of claim 42 wherein the composition comprises about 1-200 mg of caffeine.

44. The method of claim 43 wherein the composition comprises about 1-60 mg of caffeine.

45. The method of claim 36 further comprising administering the composition to the mammal at least about 30 minutes before the mammal eats a meal.

46. The method of claim 32 wherein the composition further comprises at least one of L-glutamine, chlorogenic acid, green coffee extract, lysine, inositol, choline, vitamin A, calcium, magnesium, potassium, vitamin C and quercitrin.
